(19) United States
(12) Patent Application Publication
(10) Pub. No.: US 2019/0274570 A1
(43) Pub. Date: Sep. 12, 2019

(54) ELECTROENCEPHALOGRAM DETECTION DEVICE AND EQUIPMENT

(71) Applicant: BOE TECHNOLOGY GROUP CO., LTD., Beijing (CN)

(72) Inventors: Lin ZHU, Beijing (CN); Xinguo LI, Beijing (CN)

(73) Assignee: BOE TECHNOLOGY GROUP CO., LTD., Beijing (CN)

(21) Appl. No.: 16/064,664

(22) PCT Filed: Oct. 25, 2017
(86) PCT No.: PCT/CN2017/107573

§ 371 Date: Jun. 21, 2018

(30) Foreign Application Priority Data
May 8, 2017 (CN) 201710316760.7

(51) Int. Cl. A61B 5/0478; A61B 5/04; A61B 5/00

(52) U.S. Cl. CPC A61B 5/0478; A61B 2560/0214; A61B 5/0006; A61B 5/04012

(57) ABSTRACT

An electroencephalogram detection device is disclosed includes an electroencephalogram collecting unit and an electroencephalogram processing unit. The electroencephalogram collecting unit is configured to collect brain wave signals, and then send the brain wave signals to the electroencephalogram processing unit; the electroencephalogram processing unit is configured to receive the brain wave signals, and analyze the brain wave signals; the electroencephalogram collecting unit includes a self-powered module for supplying power to it.

### TECHNICAL FIELD

Embodiments of the present disclosure relate to an electroencephalogram detection device and an electroencephalogram detection equipment.

### BACKGROUND

The human body's tissue cells are always producing very weak bioelectrical activities spontaneously and constantly. A brain wave signal is the overall effect of electrical activities of a large number of cerebral neurocytes in a highly coherent state on a pallium. If brain wave signals are collected by using an electrode that is placed on a scalp, amplified by an electroencephalogram detection equipment and recorded on a special paper, then a graph or curve with certain waveform, amplitude, frequency and phase (i.e. an encephalogram) can be obtained.

The way to get an encephalogram at present is to acquire brain wave signals by means of using a subcutaneous electrode. For example, one end of the electrode reaches into the brain, so as to detect brain wave signals at a specific location, and the other end of the electrode is connected with a processing circuit. Because the processing circuit lies outside the human brain, it is necessary to connect a wire to the processing circuit for charging when there is a need to charge the electrode and this is very inconvenient.

### SUMMARY

In view of this, an objective of embodiments of the present disclosure is to provide an electroencephalogram detection device and equipment, in which, charging of an electrode can be achieved without the need of connecting a wire.

In order to achieve the objective above, an electroencephalogram detection device is provided in the embodiments of this disclosure, comprising an electroencephalogram collecting unit and an electroencephalogram processing unit; the electroencephalogram collecting unit is configured to collect brain wave signals, and then send the brain wave signals to the electroencephalogram processing unit; the electroencephalogram processing unit is configured to receive the brain wave signals, and analyze the brain wave signals; wherein the electroencephalogram collecting unit includes a self-powered module for supplying power to the electroencephalogram collecting unit.

For example, the self-powered module includes an inductance coil, which is configured to generate a current under the action of an external magnetic field, so as to supply power for the electroencephalogram collecting unit.

For example, an outside of the electroencephalogram processing unit is surrounded by the inductance coil.

For example, the electroencephalogram collecting unit further includes an electrode, which is configured to collect brain wave signals in a preset region of the human brain.

For example, the electroencephalogram collecting unit further includes a processing circuit, which is configured to amplify the brain wave signals collected by the electroencephalogram collecting unit.

For example, the electroencephalogram collecting unit further includes a first wireless transceiving module, which is configured to send the amplified brain wave signals to the electroencephalogram processing unit.

For example, the first wireless transceiving module includes a first Bluetooth module.

For example, the electroencephalogram collecting unit further includes a housing, which is configured to encapsulate the electroencephalogram collecting unit, and moreover to expose one end of the electrode out of the housing, for ease of collecting brain wave signals.

For example, on the housing, there is also provided a through hole, the electrode protrudes from the through hole, and forms signal connection with cranial nerves.

For example, the electroencephalogram processing unit includes a central control module, an electrical source module and a second wireless transceiving module; the central control module is configured to receive amplified brain wave signals that are sent by the electroencephalogram collecting unit, and to process the amplified brain wave signals; the electrical source module is configured to supply power for the central control unit; and the second wireless transceiving module is configured to receive the amplified brain wave signals that are sent by the electroencephalogram collecting unit.

For example, the second wireless transceiving module includes a second Bluetooth module.

For example, the central control module is also configured to send a control instruction for adjusting brain wave signals to the electroencephalogram collecting unit in a preset condition.

An electroencephalogram detecting equipment comprising the electroencephalogram detection device is also provided in the embodiments of this disclosure, and the electroencephalogram detecting equipment further comprises an external charging unit for supplying power to the electroencephalogram detection device and an external equipment connected to the electroencephalogram detection device.

For example, the external equipment comprising at least one of the following: a display screen and a control center; when the external equipment is the display screen, the electroencephalogram processing unit is configured to send the brain wave signals to the display screen for display; when the external equipment is the control center, the electroencephalogram processing unit is configured to send the amplified brain wave signals to the control center for processing, and to receive an instruction sent by the control center.

A technical solution according to an embodiment of the present disclosure includes an electroencephalogram collecting unit and an electroencephalogram processing unit, wherein the electroencephalogram collecting unit includes a self-powered module for supplying power to it, thus avoiding the problem brought about by supplying power to the electroencephalogram collecting unit by means of connecting a wire.

### BRIEF DESCRIPTION OF THE DRAWINGS

FIG. 1 is a schematic view showing an electroencephalogram detection device according to Embodiment 1 of the present disclosure;

FIG. 2 is a schematic view showing an electroencephalogram collecting unit according to Embodiment 2 of the present disclosure;

FIG. 3 is a schematic view showing the power supply of a transmitting coil and an electroencephalogram collecting unit of an electroencephalogram detection device according to Embodiment 2 of the present disclosure;

FIG. 4 is a schematic view showing the connection between an inductance coil and a processing circuit of the electroencephalogram detection device according to Embodiment 2 of the present disclosure;

FIG. 5 is a structurally schematic view showing the electroencephalogram collecting unit of the electroencephalogram detection device according to Embodiment 2 of the present disclosure;

FIG. 6 is a schematic view showing implantation of the electroencephalogram collecting unit of the electroencephalogram detection device into a human brain according to Embodiment 2 of the present disclosure; and

FIG. 7 is a schematic view showing the connection between the electroencephalogram detection device and an external charging unit as well as an external equipment according to Embodiment 2 of the present disclosure.

### DESCRIPTION

Thereinafter, specific embodiments of the present disclosure will be further described in detail in connection with the accompanied drawings and embodiments. The following embodiments are used to explain the present disclosure, but are not used to limit the scope of the present disclosure.

### Embodiment 1

FIG. 1 is a schematic view showing an electroencephalogram detection device according to Embodiment 1 of the present disclosure, and as shown in FIG. 1, an electroencephalogram detection device according to the embodiment, includes an electroencephalogram collecting unit 10 and an electroencephalogram processing unit 20. In embodiments of the present invention, the electroencephalogram collecting unit 10, for example, may include a sensor, an electrode and so on. The electroencephalogram processing unit, for example, may be a microprocessor chip, a general-purpose processor (e.g., a central processor), or a specialized processor (e.g., a programmable logic circuit).

The electroencephalogram collecting unit 10 is configured to collect brain wave signals, and concretely, it is possible that according to the actually adopted electroencephalogram acquiring equipment, for example, it may be implanted into a human brain, and collect brain wave signals, and then it transmits the brain wave signals to the electroencephalogram processing unit; the electroencephalogram processing unit is configured to receive the brain wave signals, and analyze the brain wave signals; the electroencephalogram collecting unit 10 includes a self-powered module 101 for supplying power to it.

The self-powered technology is a new power supply technology, and it transforms all kinds of energy in the surrounding environment into electrical energy, so as to drive electronic devices with low power consumption to operate. By utilizing the self-powered technology, zero power consumption can be effectively achieved, thus saving the installation and use costs and protecting the environment. The self-powered module 101 according to the embodiment can act to convert the surrounding energy into electrical energy and provide energy for the electroencephalogram collecting unit 10. For example, upon implementation, an inductance coil 101 may be used as the self-powered module 101.

Since it is unnecessary for the self-powered module 101 to be connected to an externally connected power supply, the electroencephalogram collecting unit 10 according to the embodiment does not need an additional source for power supply, either. Consequently, when the electroencephalogram collecting unit 10 acts to detect brain wave, it is unnecessary for it to be connected externally to an extra wire for power supply. In this way, not only such an issue that the electroencephalogram collecting unit 10 is exposed to outside of the human brain and thus an electrode 102 is oxidized easily can be avoided, but also such a problem that the electroencephalogram collecting unit 10 needs to be replaced frequently owing to oxidation can be avoided. This brings great convenience to the patients in need of electroencephalogram detection.

The technical solution according to an embodiment of the present disclosure includes an electroencephalogram collecting unit 10 and an electroencephalogram processing unit 20, the electroencephalogram collecting unit 10 includes a self-powered module 101 for supplying power to it, thus avoiding the problem brought about by supplying power to the electroencephalogram collecting unit 10 by means of connecting a wire.

### Embodiment 2

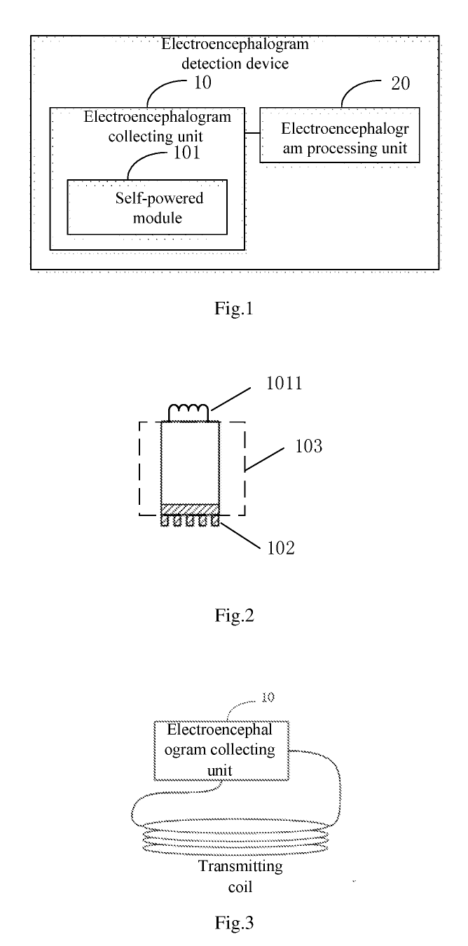

FIG. 2 is a schematic view showing an electroencephalogram collecting unit 10 according to Embodiment 2 of the present disclosure. As shown in FIG. 2, in a specific embodiment, the self-powered module 101 includes an inductance coil 1011, which is configured to generate a current under the action of an external magnetic field, so as to supply power for the electroencephalogram collecting unit 10.

For example, the self-powered principle of the inductance coil 1011 can be described briefly as follows: when a transmitting coil gets close to the inductance coil 1011, a varied magnetic field can be formed on the inductance coil 1011 at the moment of energization, and a current is formed by the inductance coil 1011 in the varied magnetic field. A power supply circuit including the transmitting coil is shown in FIG. 3. It is to be noted that, the transmitting coil is connected to the electroencephalogram collecting unit 10 with a solid line, and this is only to show that the two have the power supply relationship. Upon the concrete implementation, the two are not in a relationship of direct connection, but in a non-contact power supply mode.

Further, in conjunction with FIG. 2 continually, the electroencephalogram collecting unit 10 further includes an electrode 102. It is possible that according to the actually adopted acquiring device as stated hereinbefore, the electrode 102 is configured to be implanted into a human brain, and collect brain wave signals in a preset region of the human brain.

For example, Electroencephalogram (EEG) refers to such a phenomenon that when the brain is in action, a potential difference is formed between pallium cell masses, and thus a current is produced outside cells of the pallium. It records change in wave during brain activity, and is the overall reflection of electrophysiological activities of cerebral neurocytes on the pallium or a surface of scalp. The electroencephalogram monitoring is widely used in clinical practice applications. As the electroencephalogram has the traits of low frequency and weak signal, it is required that an electrode be implanted into a human brain. In this way, the potential between two points in the brain can be recorded, so that the medical staff can observe the change in the patient's brain waves.

Further, the electroencephalogram collecting unit 10 further includes a processing unit 103 (e.g., an amplifier), and the processing unit 103 is configured to amplify the brain wave signals collected by the electroencephalogram collecting unit 10.

For example, because brain wave signals are relatively weak, and their frequency is relatively low, if the brain wave signals acquired by the electrode 102 are sent directly to the electroencephalogram processing unit 20, then they will not be able to be analyzed and processed directly. Therefore, after brain wave signals are detected by the electrode 102, they need to be amplified by the processing circuit 103.

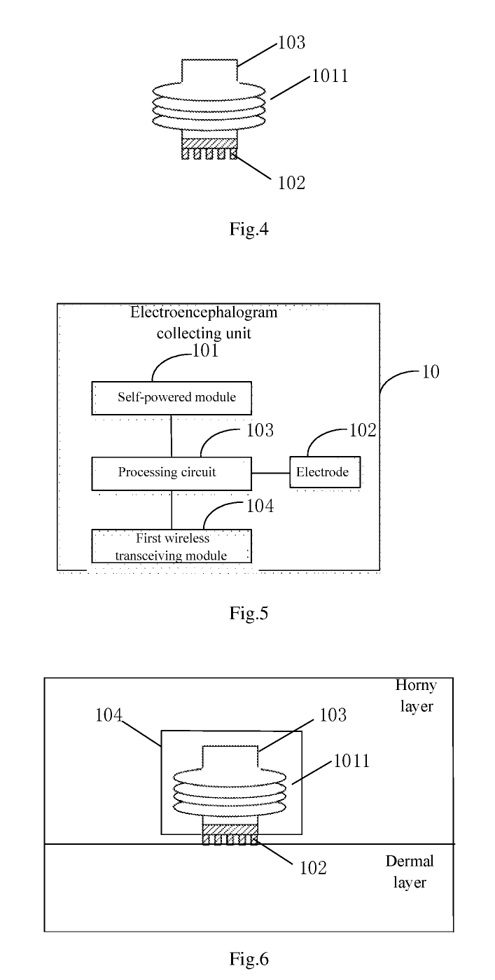

In an example, as shown in FIG. 4, outside the processing circuit can be surrounded by an inductance coil. In this way, it is possible that the space is saved, and volume of the electroencephalogram collecting unit is decreased.

Further, as shown in FIG. 5, the electroencephalogram collecting unit 10 further includes a first wireless transceiving module 104, which is configured to transmit the amplified brain wave signals to the electroencephalogram processing unit 20.

For example, data transmission can be done between the electroencephalogram collecting unit 10 and the electroencephalogram processing unit 20 in a wired or wireless manner. But if data transmission is done in a wired manner, then the case that one end of a connecting wire is implanted into the brain and the other end of it protrudes to outside of the brain will result, causing inconvenience to the patient. Therefore, according to the embodiment, in order to avoid the problem that an electroencephalogram collecting unit 10 needs to be connected with a data line upon data transmission, a wireless transmission mode is adopted as the data transmission mode, and for example, a Bluetooth mode is adopted. For example, the first wireless transceiving module 104 (e.g., a wireless signal transceiver) includes a first Bluetooth module. In other embodiments, other wireless data transmission mode, such as WIFI or the like, may also be adopted.

Further, as shown in FIG. 6, the electroencephalogram collecting unit 10 further includes a housing 104, which is configured to encapsulate the electroencephalogram collecting unit, and moreover, to expose one end of the electrode 102 to outside of the housing 104, so that it forms signal connection with cranial nerves in the form of, such as biological discharge, for ease of collection of brain wave signals.

For example, since a plurality of devices, such as the processing circuit 103, the electrode 102, the self-powered module 101 and the first wireless transceiving module, are included in the electroencephalogram collecting unit 10, for ease of protecting each component, the housing 104 may be set at the outermost layer of the electroencephalogram collecting unit 10. At the same time, a through hole is arranged on the housing 104, and thus the electrode 102 protrudes from the through hole 102, and forms signal connection with cranial nerves, so as to measure potential differences in different parts of the brain.

In conjunction with FIG. 6 continually, it shows the position of the electroencephalogram collecting unit 10 in the human brain. The electroencephalogram collecting unit is implanted into a horny layer, and since the horny layer lies at the outermost layer of the human brain, the patient's discomfort is avoided by doing this.

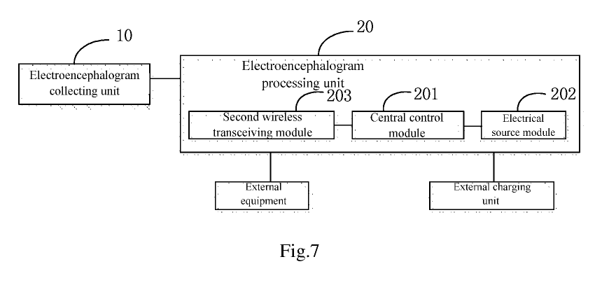

Further, as shown in FIG. 7, the electroencephalogram processing unit 20 includes a central control module 201, an electrical source module 202 and a second wireless transceiving module 203. The central control module 201 (e.g., a processor) is configured to receive amplified brain wave signals that are sent by the electroencephalogram collecting unit 10, and to process the amplified brain wave signals; the electrical source module (e.g. a battery) is configured to supply power for the central control unit; and the second wireless transceiving module 203 (e.g., a wireless transceiver) is configured to receive the amplified brain wave signals that are sent by the electroencephalogram collecting unit 10.

For example, the electroencephalogram processing unit 20 receives the amplified brain wave signals, and it may analyze the amplified brain wave signals and then present them in a relatively intuitive way. Specifically, they are processed by the central control module 201 (MCU), and the power is supplied by the electrical source module 202 to MCU. In correspondence with the electroencephalogram collecting unit 10, the electroencephalogram processing unit 20 is provided with a second wireless transceiving module (e.g., a wireless signal transceiver), and the second wireless transceiving module 203 may be a second Bluetooth module.

Further, the central control module 201 is also configured to send control instructions for adjusting brain wave signals to the electroencephalogram collecting unit 10 in a preset condition.

The preset condition may be that brain wave signals lies in a preset reference range. And the preset reference range is an abnormal reference value, and the preset reference range indicates that the patient is being in a morbid period. In an application scenario, when brain wave signals show that the patient is in a period of illness seizure, the central control module 201 may send a control instruction to the electroencephalogram collecting unit 10, for giving an electrical signal of opening video to the brain, so as to stimulate a dermal layer of the brain. For example, for an epileptic patient, at the time of epileptic seizure, by means of giving a certain stimulation to its brain with aid of an electroencephalogram collecting unit implanted into its brain, epileptic seizure can be inhibited.

The technical solution according to an embodiment of the present disclosure includes an electroencephalogram collecting unit and an electroencephalogram processing unit, the electroencephalogram collecting unit includes a self-powered module for supplying power to it, thus avoiding the problem brought about by supplying power to the electroencephalogram collecting unit by means of connecting a wire.

### Embodiment 3

According to the present embodiment, there is also provided an electroencephalogram detection equipment, which includes the electroencephalogram detection device involved in any of embodiments shown in FIG. 1 to FIG. 7, and further includes an external charging unit for supplying power to the electroencephalogram detection device and an external equipment connected to the electroencephalogram detection device. Please refer to FIG. 6 for details.

In an example, the electrical source unit may be a rechargeable battery, and in this case, an external charging unit is needed to charge it. In this way, it is avoided from being restricted to the environment such as power failure.

The external equipment includes at least one of the following: a display screen and a control center. When the external equipment is a display screen, the electroencephalogram processing unit 20 is configured to send the brain wave signals to the display screen for display. when the external equipment is a control center (e.g., a server), the electroencephalogram processing unit 20 is configured to send the amplified brain wave signals to the control center for processing, and to receive an instruction sent by the control center.

For example, in order to present the amplified brain wave signals in an intuitive way, the electroencephalogram processing unit 20 may be connected to a display screen, and in this way, brain wave signals can be presented in the manner of electroencephalogram. Moreover, in order to make a deeper analysis of the patient's condition, the amplified brain wave signals can be sent to the control center, for ease of making analysis in conjunction with other data, and also for ease of saving the patient's archival data.

The technical solution according to an embodiment of the present disclosure includes an electroencephalogram collecting unit and an electroencephalogram processing unit, the electroencephalogram collecting unit includes a self-powered module for supplying power to it, thus avoiding the problem brought about by supplying power to the electroencephalogram collecting unit by means of connecting a wire.

What are described above is related to the illustrative embodiments of the disclosure only and not limitative to the scope of the disclosure; the scopes of the disclosure are defined by the accompanying claims. Any change, equivalent replacement of the present disclosure, made within the substantial protected scope by the skilled in the art, shall all fall within the scope of protection of the present disclosure.

The present application claims priority of Chinese patent application No. 201710316760.7, filed on May 8, 2017, the entire disclosure of which is incorporated by reference herein as a part of the present application.

1. An electroencephalogram detection device, comprising an electroencephalogram collecting unit and an electroencephalogram processing unit;
wherein the electroencephalogram collecting unit is configured to collect brain wave signals, and then send the brain wave signals to the electroencephalogram processing unit; and
the electroencephalogram processing unit is configured to receive the brain wave signals, and analyze the brain wave signals; the electroencephalogram collecting unit includes a self-powered module for supplying power to the electroencephalogram collecting unit.

2. The device claimed as claim 1, wherein the self-powered module includes an inductance coil, and the inductance coil is configured to generate a current under the action of an external magnetic field, so as to supply power for the electroencephalogram collecting unit.

3. The device claimed as claim 2, wherein an outside of the electroencephalogram processing unit is surrounded by the inductance coil.

4. The device claimed as claim 1, wherein the electroencephalogram collecting unit further includes an electrode, which is configured to collect brain wave signals in a preset region of a human brain.

5. The device claimed as claim 1, wherein the electroencephalogram collecting unit further includes a processing circuit, which is configured to amplify the brain wave signals collected by the electroencephalogram collecting unit.

6. The device claimed as claim 5, wherein the electroencephalogram collecting unit further includes a first wireless transceiving module, which is configured to send the brain wave signals that are amplified to the electroencephalogram processing unit.

7. The device claimed as claim 6, wherein the first wireless transceiving module includes a first Bluetooth module.

8. The device claimed as claim 4, wherein the electroencephalogram collecting unit further includes a housing, and the housing is configured to encapsulate the electroencephalogram collecting unit, and moreover to expose one end of the electrode out of the housing for ease of collecting brain wave signals.

9. The device claimed as claim 8, wherein, on the housing, there is also provided a through hole, the electrode protrudes from the through hole, and forms signal connection with cranial nerves.

10. The device claimed as claim 1, wherein the electroencephalogram processing unit includes a central control module, an electrical source module and a second wireless transceiving module;
the central control module is configured to receive amplified brain wave signals that are sent by the electroencephalogram collecting unit, and to process the amplified brain wave signals;
the electrical source module is configured to supply power for the central control unit; and
the second wireless transceiving module is configured to receive the amplified brain wave signals that are sent by the electroencephalogram collecting unit.

11. The device claimed as claim 10, wherein the second wireless transceiving module includes a second Bluetooth module.

12. The device claimed as claim 10, wherein the central control module is also configured to send a control instruction for adjusting brain wave signals to the electroencephalogram collecting unit in a preset condition.

13. An electroencephalogram detecting equipment comprising the electroencephalogram detection device claimed as claim 1, and further comprising an external charging unit for supplying power to the electroencephalogram detection device and an external equipment connected to the electroencephalogram detection device.

14. The equipment claimed as claim 13, wherein the external equipment comprising at least one of a display screen and a control center;
in a case where the external equipment is the display screen, the electroencephalogram processing unit is configured to send the brain wave signals to the display screen for display; and
in a case where the external equipment is the control center, the electroencephalogram processing unit is configured to send the amplified brain wave signals to the control center for processing, and to receive an instruction sent by the control center.

15. The device claimed as claim 5, wherein the electroencephalogram processing unit includes a central control module, an electrical source module and a second wireless transceiving module;
the central control module is configured to receive amplified brain wave signals that are sent by the electroencephalogram collecting unit, and to process the amplified brain wave signals;
the electrical source module is configured to supply power for the central control unit; and
the second wireless transceiving module is configured to receive the amplified brain wave signals that are sent by the electroencephalogram collecting unit.

16. The device claimed as claim 15, wherein the second wireless transceiving module includes a second Bluetooth module.

17. The device claimed as claim 15, wherein the central control module is also configured to send a control instruction for adjusting brain wave signals to the electroencephalogram collecting unit in a preset condition
